(19) United States
(12) Patent Application Publication
(10) Pub. No.: US 2005/0112123 A1
(43) Pub. Date: May 26, 2005

(54) Methods of treating proteinuria

(76) Inventors: Michael R. Vaughan, Seattle, WA (US); Stuart J. Shankland, Seattle, WA (US); Leah Haseley, Seattle, WA (US); Jeffrey W. Pippin, Seattle, WA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/960,197

(22) Filed: Oct. 6, 2004

Related U.S. Application Data
(60) Provisional application No. 60/508,929, filed on Oct. 6, 2003.

(51) Int. Cl. A61K 38/13; A61K 31/66; A61K 39/395; A61K 31/573; A61K 31/4745; A61K 31/203; A61K 31/202

(52) U.S. Cl. 424/144.1; 514/559; 514/423; 514/11; 514/109; 514/171; 514/449; 514/291; 514/560

(57) ABSTRACT

The present invention provides methods of treating proteinuria, and treating renal disorders associated with proteinuria, the methods generally involving administering to an individual having such a disorder an effective amount of retinoic acid receptor agonist or a retinoid X receptor agonist.

### CROSS-REFERENCE

This application claims the benefit of U.S. Provisional Patent Application No. 60/508,929 filed Oct. 6, 2003, which application is incorporated herein by reference in its entirety.

### STATEMENT REGARDING FEDERALLY SPONSORED RESEARCH

The U.S. government may have certain rights in this invention, pursuant to grant number DK60525 awarded by the National Institutes of Health.

### FIELD OF THE INVENTION

The present invention is in the field of treating proteinuria, and in particular renal disorders associated with proteinurea.

### BACKGROUND OF THE INVENTION

In a healthy kidney, the glomerular capillary wall acts as a barrier to prevent proteins from entering the urine, based on size and electrical charge of the proteins. The primary barrier for ultrafiltration of plasma in renal glomeruli comprises three layers; a fenestrated endothelium, a 300-350 nm thick glomerular basement membrane, and slit pores, i.e. diaphragms located between the foot processes of the epithelial cells. If the glomerulus is intact, only trace amounts of albumin escape in the glomerular filtrate. Proteins that are smaller than albumin (e.g., proteins having a molecular weight of less than about 68,000 daltons) are filtered and reabsorbed by the proximal tubule. Under normal physiological conditions, up to 1500 mg of protein is filtered every 24 hours. Most of this is reabsorbed by the proximal tubule, where the protein undergoes catabolism. The glomerular filter is affected in a large number of acquired and inherited diseases, resulting in extensive leakage of plasma albumin and larger proteins, and leading to protein in the urine and progressive kidney disease. Nephrotic syndrome is an example of a disease characterized by proteinuria.

Over 20 million Americans suffer from kidney disease. One important determinant in a given persons rate of decline of kidney function is the amount of protein in the urine or proteinuria. Typically, patients with greater amounts of proteinuria have faster progression of kidney disease. For this reason, many efforts have been made to design treatments that would decrease the amount of proteinuria. Amongst patients who have proteinuria, there is a broad spectrum in the amount of protein that a person can have in the urine. At the higher end of the spectrum is when the amount of protein in the urine exceeds 3.5 grams in 24 hours. At this range, it is termed nephrotic range proteinuria. It is difficult to determine the prevalence of nephrotic range proteinuria. The prevalence of nephrotic range proteinuria is difficult to establish in adults because the condition is usually a result of an underlying disease.

About two in every 10,000 people in the United States experience nephrotic syndrome. Nephrotic syndrome is a constellation of signs and symptoms including proteinuria, hypoalbuminemia, edema (especially in the legs and feet), and hypercholesterolemia. Disorders that can lead to nephrotic syndrome include diabetes and hypertension. Disorders that can cause specific damage to the glomeruli, and that often result in the development of heavy proteinuria and in many instances nephrotic syndrome, include amyloidosis, congenital nephrosis, Focal segmental glomerular sclerosis (FSGS), glomerulonephritis, IgA nephropathy, minimal change disease, and pre-eclampsia. Current treatments include angiotensin converting enzyme inhibitors, and prednisone.

Despite the availability of treatments for proteinuria, there is an ongoing need in the field for treatment methods that reduce proteinuria, including nephrotic-range proteinuria. The present invention addresses this need.

Literature

Suzuki et al. (2003) J. Am. Soc. Nephrol. 14:981-991; U.S. Patent Application Publication No. 2004/0106155; U.S. Pat. No. 6,355,669; U.S. Pat. No. 6,586,476; Mundel and Shankland (2002) J. Am. Soc. Nephrol. 13:3005-3015; U.S. Pat. No. 5,236,933; U.S. Pat. No. 6,207,811; U.S. Pat. No. 5,238,924; U.S. Patent Application Publication No. 2004/0014772; and U.S. Pat. No. 6,653,322.

### SUMMARY OF THE INVENTION

The present invention provides methods of treating proteinuria, and treating renal disorders associated with proteinuria, the methods generally involving administering to an individual having such a disorder an effective amount of retinoic acid receptor agonist or a retinoid X receptor agonist.

### BRIEF DESCRIPTION OF THE DRAWINGS

FIG. 1 depicts the effect of ATRA on rat podocyte proliferation in vitro.

FIG. 2 depicts a protein blot analysis of the effect of ATRA on nephrin and podocin protein levels in mouse podocytes in vitro.

FIG. 3 depicts the effect of ATRA on proteinuria in a mouse model of podocyte injury.

FIG. 4 depicts the effect of ATRA on the number of proliferating cell nuclear antigen-positive cells per glomerulus. The results are shown for normal mice; a mouse model of podocyte injury treated with vehicle (control); and a mouse model of podocyte injury treated with ATRA.

FIG. 5 depicts the effect of ATRA on podocyte number. Podocyte number was evaluated quantitating the number of cells expressing WT-1. The results are shown for normal mice; a mouse model of podocyte injury treated with vehicle (control); and a mouse model of podocyte injury treated with ATRA at days 5 and 14.

FIG. 6 depicts the effect of ATRA on proliferation of wild-type (WT) podocytes and HIV transgenic (Tg) podocytes in vitro.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans; rodents, including mice and rats; sports animals such as horses; farm animals such as goats, sheep, and cows. Of particular interest in many embodiments is the treatment of humans.

As used herein, the phrase “glomerular disorder” includes any glomerular disease and/or any glomerular injury such as, for example, minimal change disease, focal segmental glomerular sclerosis (FSGS), membranous nephropathy, diabetic nephropathy, amyloidosis, IgA nephropathy, anti-glomerular basement membrane (anti-GBM) antibody disease, pre-eclampsia, nephrotic syndrome, infection-related renal disease, and congenital nephrosis.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a retinoic acid agonist” includes a plurality of such agonists and reference to “the active agent” includes reference to one or more active agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating proteinuria, and renal disorders associated with proteinuria, in an individual having proteinuria or a renal disorder associated with proteinuria. The methods generally involve administering to the individual an effective amount of a retinoic acid receptor (RAR) agonist or a retinoid X receptor (RXR) agonist.

In many embodiments, an effective amount of an RAR agonist or an RXR agonist is an amount that is effective to reduce the amount of protein excreted into the urine over a 24-hour period by at least about 10%, at least about 20%, at least about 25%, at least about 30%, at least about 40%, at least about 50%, at least about 60%, at least about 70%, or at least about 80%, or more, compared to the amount of protein excreted into the urine over a 24-hour period in the absence of treatment. In some embodiments, an effective amount of an RAR agonist or an RXR agonist is an amount that is effective to reduce the amount of protein excreted into the urine over a 24-hour period to within a normal range, e.g., less than about 150 mg over a 24-hour period. The amount of protein excreted in urine is readily determined using any known method. Typically, urine excreted by an individual is collected over a 24-hour period, and the total amount of protein in the 24-hour urine sample is measured. For example, urine collected in a 24-hour period is filtered, precipitated by 5% (final concentration) trichoracetic acid (TCA), collected by centrifugation, and measured by the method of Lowry et al. ((1951) Protein measurement with the Folin phenol reagent. J. Biol. Chem. 193:265-275). Another suitable method for measuring urine protein is the sulphosalicylic acid (SSA) method (see, e.g., Shankland et al. (1996) Kidney Int. 50:116-124). A variety of commercially available kits for measuring protein in urine are also suitable for use in determining 24-hour urine protein. The 24-hour protein can also be estimated by measuring protein in the urine taken during a time period of less than 24 hours, then multiplying it out such that it is equivalent to 24 hours. For example, the protein content in urine taken over a 1-hour time period is multiplied by 24 to give the 24-hour urine protein; the protein content in urine taken over a 2-hour time period is multiplied by 12 to give the 24-hour urine protein; etc. In addition, the 24-hour protein excretion can be estimated by taking a ratio of a spot urine protein and spot urine creatinine.

In some embodiments, an effective amount of an RAR agonist or an RXR agonist is an amount that is effective to reduce podocytes proliferation by at least about 10%, at least about 20%, at least about 25%, at least about 30%, at least about 40%, at least about 50%, at least about 60%, at least about 70%, or at least about 80%, or more, compared to the level of podocytes proliferation in the absence of treatment. Whether a subject method reduces the level of podocytes proliferation can be determined using any known method. For example, podocytes can be cultured in vitro in the presence of an RAR agonist or an RXR agonist, and the level of proliferation, compared to the level of proliferation of podocytes cultured in vitro in the absence of the RAR agonist or the RXR agonist, is determined using e.g., a radioactive thymidine uptake assay.

In some embodiments, an effective amount of an RAR agonist or an RXR agonist is an amount that is effective to increase the level of a podocyte-specific polypeptide in a podocyte by at least about 10%, at least about 20%, at least about 25%, at least about 30%, at least about 40%, at least about 50%, at least about 60%, at least about 70%, at least about 80%, at least about 2-fold, at least about 5-fold, or at least about 10-fold, or more, compared to the level of the podocyte-specific polypeptide in a podocyte in the absence of treatment. Whether an RAR agonist or an RXR agonist increases the level of a podocyte-specific polypeptide in a podocyte is readily determined using any known assay, e.g., a histochemical assay, an enzyme-linked immunosorbent assay (ELISA), or a protein blot assay, using antibody specific for the podocyte-specific polypeptide. Podocyte-specific polypeptides are known in the art, and include slit diaphragm proteins nephrin, podocin, and synaptopodin; the apical membrane protein ezrin; and the podocyte transcription regulator protein WT-1. See, e.g., Mundel and Shankland (2002) J. Am. Soc. Nephrol. 13:3005-3015.

In some embodiments, an effective amount of an RAR agonist or an RXR agonist is an amount that is effective to reduce disease-induced decreases in one or more of nephrin, podocin, and synaptopodin in a podocyte by at least about 10%, at least about 20%, at least about 25%, at least about 30%, at least about 40%, at least about 50%, at least about 60%, at least about 70%, at least about 80%, at least about 2-fold, at least about 5-fold, or at least about 10-fold, or more, compared to the disease-induced decrease in the absence of treatment with the RAR agonist or the RXR agonist. The levels of nephrin, podocin, and synaptopodin in a podocyte is is readily determined using any known assay, e.g., a histochemical assay, an ELISA, or a protein blot assay, using antibody specific for nephrin, podocin, or synaptopodin, as appropriate.

In some embodiments, an effective amount of an RAR agonist or an RXR agonist is an amount that is effective to increase renal function by at least about 10%, at least about 20%, at least about 25%, at least about 30%, at least about 40%, at least about 50%, at least about 60%, at least about 70%, at least about 80%, at least about 2-fold, at least about 5-fold, or at least about 10-fold, or more, compared to renal function in the absence of treatment with the RAR agonist or the RXR agonist.

In some embodiments, a subject method comprises administration of an RAR agonist and an RXR agonist. In some embodiments, a subject method comprises administration of two or more different RAR agonists. In some embodiments, a subject method comprises administration of two or more different RXR agonists. In some of these embodiments, a subject method comprises administration of a third therapeutic agent, where the third therapeutic agent is other than an RXR agonist, and is other than an RAR agonist.

In some embodiments, a subject method comprises administration of an RAR agonist and at least one additional therapeutic agent other than an RAR agonist or an RXR agonist. In some embodiments, a subject method comprises administration of an RXR agonist and at least one additional therapeutic agent other than an RAR agonist or an RXR agonist.

Disorders Characterized by Proteinuria

The present invention provides methods for treating disorders resulting from, characterized by, or associated with, proteinuria. Proteinuria includes mild proteinuria (e.g., excretion of from about 150 mg to about 500 mg of protein in the urine over a 24-hour period); moderate proteinuria (e.g., excretion of from about 500 mg to about 1000 mg of protein in the urine over a 24-hour period); heavy proteinuria (e.g., excretion of from about 1000 mg to about 3000 mg of protein in the urine over a 24-hour period); and nephrotic range proteinuria (e.g., excretion of more than 3500 mg of protein in the urine over a 24-hour period).

Disorders that can be treated with a subject method include, but are not limited to, a glomerular disease, minimal change disease, focal segmental glomerular sclerosis (FSGS), membranous nephropathy, diabetic nephropathy, amyloidosis, IgA nephropathy, anti-glomerular basement membrane (anti-GBM) antibody disease, pre-eclampsia, nephrotic syndrome, human immunodeficiency virus (HIV) nephropathy, infection-related kidney disease, post-infection glomerulonephritis, lupus nephritis, and congenital nephrosis.

Retinoic Acid Receptor Agonists

Suitable retinoic acid receptor agonists for use in a subject method include, but are not limited to, all-trans retinoic acid (ATRA); selective retinoic acid receptor agonists such as (CD271=adapalene, an RAR-β,γ agonist; CD2043, an RAR pan-agonist; and CD336=Am580, an RAR-α agonist; 1,25-dihydroxyvitamin D3 (1,25(OH)2D3), and analogs of 1,25-dihydroxyvitamin D3 such as 1,25(OH)2-16-ene-23-yne-D3; AGN195183; AGN190168 (tazarotene); 6-3-(1-adamantyl)-4-hydroxyphenyl)-2-naphthanoic acid, (E)-4-(1-hydroxy-1-(5,6,7,8-tetrahydro-5,5,8,8-tetramethyl-2naphthyl)-2-pr openyl)benzoic acid, 4-[(E)-2-(3-(1-adamantyl)-4-hydroxyphenyl)-1-propenyl]benzoic acid, 5′,5′,8′,8′-tetramethyl-5′,6′,7′,8′-tetrahydro-[2,2′]binaphthalenyl-6-carboxylic acid, 2-(5,5,8,8-tetramethyl-5,6,7,8-tetrahydro-naphthalen-2-yl)-benzo[b]thiophene-6-carboxylic acid, 4-(5,5,8,8-tetramethyl-5,6,7,8-tetrahydro-naphtho[2,3-b]thiophen-2-yl)benzoic acid, 6-(5,5,8,8-tetramethyl-5,6,7,8-tetrahydro-naphthalene-2-carbonyl)naphthalene-2-carboxylic acid, 3,7-dimethyl-7-(1,2,3,4-tetrahydro-1,4a,9b-trimethyl-1,4-methano-dibenzofuran-8-yl)-2,4,6-heptatrienoic acid, 6-(1,2,3,4-tetrahydro-1,4a,9b-trimethyl-1,4-methano-dibenzofuran-8-yl)-naphthalene-2-carboxylic acid, 6-[hydroxyimino-(5,5,8,8-tetramethyl-5,6,7,8-tetrahydro-naphthalen-2-yl)-methyl]naphthalene-2-carboxylic acid, 4-[(6-hydroxy-7-(1-adamantyl)-2-naphthyl]benzoic acid, 5-(5,5,8,8-tetramethyl-5,6,7,8-tetrahydro-anthracen-2-yl)-thiophene-2-carboxylic acid, (−)-6-[hydroxy-(5,5,8,8-tetramethyl-5,6,7,8-tetrahydro-naphthalen-2-yl)-me thyl]-naphthalene-2-carboxylic acid, 6-(3-adamantan-1-yl-4-prop-2-ynyloxy-phenyl)-naphthalene-2-carboxylic acid, 4-[(2-oxo-2-(5,5,8,8-tetramethyl-5,6,7,8-tetrahydro-naphthalen-2-yl)-ethoxy]-benzoic acid, 4-[2-oxo-2-(5,5,8,8-tetramethyl-5,6,7,8-tetrahydro-naphthalen-2-yl)-acetyl amino]-benzoic acid, 4-[2-fluoro-2-(5,5,8,8-tetramethyl-5,6,7,8-tetrahydro-naphthalen-2-yl)-ace tylamino]-benzoic acid, 6-[3-(1-adamantyl-4-(2-hydroxypropyl)phenyl]-2-naphthoic acid, 5-[3-oxo-3-(5,5,8,8-tetramethyl-5,6,7,8-tetrahydro-naphthalen-2-yl)-propenyl]-thiophene-2-carboxylic acid, 6-[3-(1-adamantyl-4-(2,3-di-hydroxypropyl)phenyl]-2-naphthoic acid, 4-[3-hydroxy-(5,6,7,8-tetrahydro-5,5,8,8-tetramethyl-2-naphthyl)-1-propynyl]-benzoic acid, 4-[3-oxo-3-(5,5,8,8-tetramethyl-5,6,7,8-tetrahydro-naphthalen-2-yl)-prop-1-ynyl]benzoic acid, 4-[(3-(1-methylcyclohexyl)-4-hydroxyphenyl)ethenyl]-benzoic acid, 4-[(E)₂-(3-(1-adamantyl)-4-hydroxyphenyl)-ethenyl]-benzoic acid, 4-[3-(1-adamantyl)-4-hydroxyphenylethynyl)-benzoic acid, 5-[3-(1-adamantyl)-4-hydroxyphenylethynyl]-2-thiophenecarboxylic acid, 5-[3-(1-adamantyl)-4-methoxyphenylethynyl]-2-thiophene-carboxylic acid, 4-[2-(3-tert-butyl-4-methoxyphenyl)-propenyl]benzoic acid, 4-{2-[4-methoxy-3-(1-methyl-cyclohexyl)phenyl]-propenyl}-benzoic acid, 6-[3-(1-adamantyl)-4-(3-methoxy-2-hydroxypropyl)-phenyl]-2-naphthoic acid, 2-hydroxy-4-[3-hydroxy-3-(5,6,7,8-tetrahydro-5,5,8,8-tetramethyl-2-naphthyl)-1-propynyl]-benzoic acid, 6-(5,5,8,8-tetramethyl-5,6,7,8-tetrahydro-naphthalen-2-yloxy)-naphthalene-2-carboxylic acid, 6-(5,5,8,8-tetramethyl-5,6,7,8-tetrahydro-naphthalen-2-ylsulphanyl)-naphth alene-2-carboxylic acid, 4-[2-propoxyimino-2-(5,5,8,8-tetramethyl-5,6,7,8-tetrahydro-naphthalen-2-yl)-acetylamino]benzoic acid, 6-(5,5,8,8-tetramethyl-5,6,7,8-tetrahydro-naphthalen-2-ylamino)naphthalene-2-carboxylic acid, 1-methyl-4-(5,5,8,8-tetramethyl-5,6,7,8-tetrahydroanthracen-2-yl)-1H-pyrrole-2-carboxylic acid, 2-methoxy-4-(5,5,8,8-tetramethyl-5,6,7,8-tetrahydro-anthracen-2-yl)-benzoic acid, 4-[2-nonyloxyimino-2-(5,5,8,8-tetramethyl-5,6,7,8-tetrahydro-naphthalen-2-yl)-acetylamino]-benzoic acid, (−)-2-hydroxy-4-[3-hydroxy-3-(5,5,8,8-tetramethyl-5,6,7,8-tetrahydro-naphthalen-2-yl)-prop-1-ynyl]-benzoic acid, (+)-2-hydroxy-4-[3-hydroxy-3-(5,5,8,8-tetramethyl-5,6,7,8-tetrahydro-naphthalen-2-yl)-prop-1-ynyl]-benzoic acid, 2-hydroxy-4-[3-hydroxy-3-(5,5,8,8-tetramethyl-5,6,7,8-tetrahydro-naphthalen-2-yl)-but-1-ynyl]-benzoic acid, 6-(3-bromo-5,5,8,8-tetramethyl-5,6,7,8-tetrahydro-naphthalen-2-yloxy)-naphthalene-2-carboxylic acid, 3-[(5,6,7,8-tetrahydro-5,5,8,8-tetramethyl-2-naphthyl)-2H-1-benzopyran]-7-carboxylic acid, 4-[3-(3,5-di-tert-butyl-4-hydroxy-phenyl)-prop-1-ynyl]-benzoic acid, 4-[3-(5,5,8,8-tetra-methyl-5,6,7,8-tetrahydro-naphthalen-2-yl)-prop-1-ynyl]-benzoic acid, 4-[3-(5,6,7,8-tetrahydro-5,5,8,8-tetramethyl-2-naphthyl)-1-propynyl]-salicylic acid, 4-[{3-(1-adamantyl)-4-(2-hydroxyethyl)phenyl}ethynyl]-benzoic acid and 4-[{3-(1-adamantyl)-4-(3-hydroxy-propyl)phenyl}ethynyl]-benzoic acid.

Other suitable RAR agonists include synthetic retinoids such as Am580; Compound 1 and Compound 2 (the structures of which are disclosed in Ostrowski et al., Proc. Natl. Acad. Sci. USA 92:1812-1816 (1995; Am80 (Roy et al., Mol. Cell. Biol. 15(12):6481-6487 (1995); a RAR agonist as discussed in U.S. Pat. No. 6,653,322;
In particular embodiments of interest, the RAR agonist is ATRA. ATRA is available as Atragen®, Avita®, Renova®, Retin-A®, Vesanoid®, Vitinoin®, Lipo ATRA, Tretinoin Liposomal, AR-623, and Tretinoin®.
   Retinoid X Receptor Agonists

RXR agonists that are suitable for use in a subject method include, but are not limited to, 9-cis retinoic acid; synthetic retinoids such as SR11237 (the structure of which is disclosed in Lehman, J. M., et al., Science 258:1944-1946 (1992); LG1069, the structure and preparation of which are described in Boehm et al., J. Med. Chem. 37:2930-2941 (1994); an RXR agonist as described in U.S. Pat. No. 6,610,883; an RXR agonist as described in U.S. Pat. No. 6,593,493; etc.

Dosages, Formulations, and Routes of Administration

An active agent (e.g., an RAR agonist, an RXR agonist, an additional therapeutic agent such as an ACE inhibitor, a corticosteroid, etc.) is administered to individuals in a formulation with a pharmaceutically acceptable excipient(s). The terms “active agent” and “therapeutic agent” are used interchangeably herein. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent may be administered to the host using any convenient means capable of resulting in the desired therapeutic effect. Thus, an active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent can be achieved in various ways, including oral, buccal, rectal, parenteral, intraperitoneal, intradermal, subcutaneous, intramuscular, transdermal, intratracheal, etc., administration. In some embodiments, e.g., in a combination therapy as described herein, two different routes of administration are used. For example, in some embodiments, an RAR agonist is administered intravenously, while an ACE inhibitor is administered orally.

Subcutaneous administration of a therapeutic agent can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, a therapeutic agent is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally used because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump are also suitable for use with the present invention. Examples of such devices include those described in, for example, U.S. Pat. Nos. 4,692,147; 4,360,019; 4,487,603; 4,360,019; 4,725,852, and the like. In general, a subject treatment method can be carried out using any of a variety of refillable, non-exchangeable pump systems. Pumps and other convective systems are in some embodiments used due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in a subject treatment method include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted above, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a therapeutic agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent (e.g., an RAR agonist, an RXR agonist, an additional therapeutic agent such as an ACE inhibitor, a corticosteroid, etc.) is formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the agent(s) in a composition as a solution in sterile water, normal saline or another pharmaceutically acceptable carrier.

Dosages

The term “unit dosage form,” as used herein, refers to physically discrete units suitable as unitary dosages for human and animal subjects, each unit containing a predetermined quantity of an active agent (e.g., an RAR agonist, an RXR agonist, an additional therapeutic agent such as prednisone, an ACE inhibitor, etc.) calculated in an amount sufficient to produce the desired effect in association with a pharmaceutically acceptable diluent, carrier or vehicle. The specifications for the a unit dosage forms depend on the particular compound employed and the effect to be achieved, and the pharmacodynamics associated with each compound in the host.

Monotherapy

In some embodiments, a subject method involves administering to an individual in need thereof an effective amount of an RAR agonist, or an RXR agonist, as monotherapy.

In some embodiments, an RAR agonist is administered, where the RAR agonist is ATRA. In some embodiments, ATRA is administered in a dosage of from about 5 mg to about 100 mg per m² body surface per day, e.g., from about 5 mg/m² to about 10 mg/m², from about 10 mg/m² to about 15 mg/m², from about 15 mg/m² to about 20 mg/m², from about 20 mg/m² to about 25 mg/m², from about 25 mg/m² to about 30 mg/m², from about 30 mg/m² to about 35 mg/m², from about 35 mg/m² to about 40 mg/m², from about 40 mg/m² to about 45 mg/m², from about 45 mg/m² to about 40 mg/m², from about 40 mg/m² to about 60 mg/m², from about 60 mg/m² to about 70 mg/m², from about 70 mg/m² to about 80 mg/m², from about 80 mg/m² to about 90 mg/m², or from about 90 mg/m² to about 100 mg/m² per day. In many embodiments, the ATRA is administered orally. In a particular embodiment, ATRA is administered in a dosage of about 45 milligrams (mg) per m² body surface per day, administered orally in two equally divided doses.

In some embodiments, ATRA is administered in a dosage of from about 2 mg/kg body weight to about 25 mg/kg body weight, e.g., from about 2 mg/kg to about 5 mg/kg, from about 5 mg/kg to about 10 mg/kg, from about 10 mg/kg to about 15 mg/kg, from about 15 mg/kg to about 20 mg/kg, or from about 20 mg/kg to about 25 mg/kg. In some embodiments, ATRA is administered in a dosage of from about 2 mg/kg body weight to about 25 mg/kg body weight, e.g., from about 2 mg/kg to about 5 mg/kg, from about 5 mg/kg t6 about 10 mg/kg, from about 10 mg/kg to about 15 mg/kg, from about 15 mg/kg to about 20 mg/kg, or from about 20 mg/kg to about 25 mg/kg daily, for a period of time of from about 1 day to about 30 days, e.g., from about 1 day to about 7 days, from about 7 days to about 2 weeks, or from about 2 weeks to about 30 days. In other embodiments, ATRA is administered in a dosage of from about 2 mg/kg body weight to about 25 mg/kg body weight, e.g., from about 2 mg/kg to about 5 mg/kg, from about 5 mg/kg to about 10 mg/kg, from about 10 mg/kg to about 15 mg/kg, from about 15 mg/kg to about 20 mg/kg, or from about 20 mg/kg to about 25 mg/kg daily, for a period of time of from about 1 day to about 1 year, or longer, e.g., from about 1 day to about 7 days, from about 7 days to about 2 weeks, or from about 2 weeks to about 1 month, from about 1 month to about 2 months, from about 2 months to about 3 months, from about 3 months to about 6 months, or from about 6 months to about 12 months, or longer.

In many embodiments, the RAR agonist (e.g., ATRA), or the RXR agonist, is administered for a period of from about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

The RAR agonist (e.g., ATRA) or the RXR agonist is administered at various frequencies, e.g., once per month, twice per month, three times per month, every other week (qow), once per week (qw), twice per week (biw), three times per week (tiw), four times per week, five times per week, six times per week, every other day (qod), daily (qd), substantially continuously, or continuously, and over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, the RAR agonist or the RXR agonist is administered until such time as the 24-hour urine protein level is within normal range. For example, in some embodiments, an RAR agonist or an RXR agonist is administered once per day; and the level of protein excreted into the urine over a 24-hour period is measured periodically (e.g. once per week, or twice per week) during the RAR agonist or RXR agonist treatment. When the level of protein excreted in the urine over a given 24-hour period falls to 150 mg or less, administration of RAR agonist or RXR agonist will in some embodiments be discontinued.

Combination Therapies

In some embodiments, a subject method involves administering to an individual in need thereof an effective amount of an RAR agonist or an RXR agonist, and at least one additional therapeutic agent that reduces proteinuria. Suitable additional therapeutic agents include, but are not limited to, an angiotensin converting enzyme (ACE) inhibitor; an angiotensin receptor blocker (ARB); a cortisosteroid; a calcium channel antagonist (a calcium channel blocker); a cytochrome P450 inhibitor; cyclophosphamide; cyclosporine; tacrolimus; mycophenolate; azathioprine; a non-steroidal anti-inflammatory drug (NSAID); rituximab; intravenous immunoglobulin; anti-C5 monoclonal antibody; levamisole; an omega-3 fatty acid; and the like.

Suitable ACE inhibitors include, but are not limited to, ramipril (Altace®), quinapril HCl (Accupril®), captopril (Capoten®), benazapril HCl (Lotrel®, Lotensin®), trandolapril (Mavik®, Tarka®), fosinopril (Monpril®), moexipril HCl (UnivascE, Uniretic®), enalopril maleate (Vasotec®, Lexxel®, Teczem®, Vaseretic®), lisinopril (Zestrilg, Zestoretic®, Prinivil®, Prinzide®); and the like.

Orally active ACE inhibitors will be used in some embodiments, where orally active ACE inhibitors include, ramipril, enalapril, captopril, alacepril, benazepril, ceranapril, cilazapril, delapril, fosinopril, imidapril, libenzapril, lisinopril, moexipril, moveltipril, perindopril, quinapril, spirapril, zofenopril, trandolapril, BPL 36378, CS 622, FPL 63547, S 9650 and others. Orally active ACE inhibitors are described, for example, in “Pharmacology of Antihypertensive Therapeutics” (Eds. D. Ganten, P. J. Mutrow) Springer Verlag, Berlin 1990, pp. 377-480. Also suitable for use is an ACE inhibitor as described in U.S. Pat. No. 5,236,933.

Benazapril is generally administered at a dosage of 10 milligrams (mg) orally once a day at first, which is in some cases increased to 20 mg to 40 mg orally per day taken as a single dose or divided into two doses. Captopril is generally administered at a dosage of 25 mg to 100 mg orally two or three times a day. Enalapril is generally administered at a dosage of 5 mg orally once a day at first, which is in some cases increased to 10 mg to 40 mg orally a day taken as a single dose or divided into two doses. Enalapril is in some embodiments administered at a dosage of 1.25 mg intravenously every six hours. Fosinopril is generally administered at a dosage of 10 mg to 40 mg orally once a day. Lisinopril is generally administered at a dosage of 10 mg to 40 mg orally once a day. Moexipril is generally administered at a dosage of 7.5 mg orally once a day at first, which is in some cases increased to 30 mg orally a day taken as a single dose or divided into two doses.

Suitable angiotensin receptor blockers (ARB) include, but are lot limited to, candesartan (Atacand®), eprosartan (Teveten®), irbesartan (Avapro®), losartan (Cozaar®), olmesartan (Benicar®), telmisartan (Micardis®), and valsartan (Diovan).

Suitable non-steroidal anti-inflammatory drugs, include, but are not limited to, 1) the oxicams, such as piroxicam, isoxicam, tenoxicam, and sudoxicam; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone, mixtures of these non-steroidal anti-inflammatory agents may also be employed, as well as the pharmaceutically-acceptable salts and esters of these agents.

Suitable corticosteroids include, but are not limited to, Suitable steroidal anti-inflammatory agents include, but are not limited to, hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, conisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures of two or more of the foregoing.

“Cytochrome P450” (“CYP”) refers to any member/isoenzyme of the CYP superfamily, which includes the mammalian CYP 1, CYP2, CYP3 and CYP4 families (Omiecinski, et al., 1999, Toxicological Sciences 48:151-156). Thus, CYP includes the mammalian cytochrome P450 genes and their gene products, such as, P450 1A, 1A2, 1B1, 2B1, 2B2, 2B4, 2B5, 2B6,2B11, 2A6, 2C6, 2C8, 2C9, 2C11, 2C18,2C19, 2D6, 2E1, 3A4, 3A5, 3A7, 4A1 and 4B1. In some embodiments, CYP is CYP isozyme CYP2B1, which is found in glomeruli. A complete mRNA sequence and translation sequence of a human CYP2B1 is found under GenBank accession number M29874.

Suitable CYP inhibitors for use in a subject combination therapy include, but are not limited to, cimetidine, piperonyl butoxide, safrole, isosafrole, myristicinfurfylline, diethyldithiocarbamate, chlormethiazol, piperine, disulfiram, diallyl sulfide, malotilate, allylmercaptan, methylprazole, orphenadrine, arylacetylenes, clorgyline and diphenhydramine. In some embodiments, a CYP inhibitors suitable for use in a subject combination therapy is selected from: cimetidine, piperonyl butoxide, piperine, diethyldithiocarbamate, chlormethiazol, disulfiram and diallyl sulfide. In some embodiments, a CYP inhibitors useful in a subject combination therapy is selected from: cimetidine, piperonyl butoxide, piperine, diethyldithiocarbamate (DEDC), and chlormethiazol.

Cimetidine is generally administered via an oral or intravenous route at a concentration in the range of about 10 mg per kg body weight to about 50 mg per kilogram of body weight, from about 15 mg/kg to about 45 mg/kg, or from about 20 mg/kg to about 40 mg/kg. Generally cimetidine is given every six hours in a twenty four hour period.

Piperine is generally administered by an oral or intravenous route in an amount ranging from about 100 mg to about 300 mg, from about 125 mg to about 250 mg, or from about 150 mg to about 200 mg. Generally piperine is administered orally once every twenty-four hours.

The inhibitor DEDC is generally administered orally or intravenously at a concentration ranging from about 0.25 g/m² to about 2.5 g/m², e.g., from about 0.4 g/m² to about 2.0 g/m², from about 0.5 g/m² to about 1.8 g/m², and or from about 0.6 g/m² to about 1.6 g/m². Generally, DEDC is given once a day.

The inhibitor chlormethiazole is generally administered orally in an amount ranging from about 0.5 g to about 3.5 g, from about 0.75 g to about 3.0 grams, from about 1.0 g to about 2.75 grams, or from about 1.2 g to about 2.4 grams. Generally, chlormethiazole is given once every twenty-four hours.

Suitable calcium channel blockers include, but are not limited to, amlodipine (Norvasc®), bepridil (Vascor®), diltiazem (Cardizem®), felodipine (Plendil®), flunarizine, isradipine, nicardipine (Cardene®), nifedipine (Procardia®), nimodipine, and verapamil (Isoptin®, Isoptin SR®, Veralan®).

Amlopidine is generally administered at a dosage of about 5 mg to 10 mg orally once a day. Diltiazem is generally administered at a dosage of about 180 mg to 240 mg orally once a day. Felodipine is generally administered at a dosage of about 5 mg to 10 mg orally once a day. Nicardipine is generally administered at a dosage of about 20 milligrams (mg) orally three times a day. Nifepidine is generally administered at a dosage of about 10 mg orally three times a day. Verapamil is generally administered at a dosage of about 240 mg to 480 mg orally once a day.

In particular embodiments, a subject combination therapy comprises administering to an individual having a disorder associated with proteinuria effective amounts of an RAR agonist or an RXR agonist and an ACE inhibitor, where the method involves administering the RAR agonist or the RXR agonist in an amount of from about 2 mg/kg per day to about 25 mg/kg per day for the desired treatment duration; and administering the ACE inhibitor in an amount of from about 10 mg to about 100 mg orally per day for the desired treatment duration, where the ACE inhibitor is selected from ramipril, enalapril, captopril, alacepril, benazepril, ceranapril, cilazapril, delapril, fosinopril, imidapril, libenzapril, lisinopril, moexipril, moveltipril, perindopril, quinapril, spirapril, zofenopril, trandolapril. In some of these embodiments, an RAR agonist is administered, and the RAR agonist is ATRA.

In a particular embodiment, a subject combination therapy comprises administering to an individual having a disorder associated with proteinuria effective amounts of an RAR agonist and an ACE inhibitor, where the method involves administering ATRA in an amount of from about 2 mg/kg per day to about 25 mg/kg per day for the desired treatment duration; and administering the ACE inhibitor for the desired treatment duration, where the ACE inhibitor and dosage is selected from benazapril administered at a dosage of 10 milligrams (mg) orally once a day at first, which is in some cases increased to 20 mg to 40 mg orally per day taken as a single dose or divided into two doses; captopril administered at a dosage of 25 mg to 100 mg orally two or three times a day; enalapril administered at a dosage of 5 mg orally once a day at first, which is in some cases increased to 10 mg to 40 mg orally a day taken as a single dose or divided into two doses; enalapril administered at a dosage of 1.25 mg intravenously every six hours; fosinopril administered at a dosage of 10 mg to 40 mg orally once a day; lisinopril administered at a dosage of 10 mg to 40 mg orally once a day; and moexipril administered at a dosage of 7.5 mg orally once a day at first, which is in some cases increased to 30 mg orally a day taken as a single dose or divided into two doses, for the desired treatment duration.

In particular embodiments, a subject combination therapy comprises administering to an individual having a disorder associated with proteinuria effective amounts of an RAR agonist and a CYP inhibitor, where the method involves administering the RAR agonist in an amount of from about 2 mg/kg per day to about 25 mg/kg per day for the desired treatment duration; and administering the CYP inhibitor in an amount of from about 10 mg to about 500 mg orally per day for the desired treatment duration, where the CYP inhibitor is selected from cimetidine, piperonyl butoxide, safrole, isosafrole, myristicinfurfylline, diethyldithiocarbamate, chlormethiazol, piperine, disulfiram, diallyl sulfide, malotilate, allylmercaptan, methylprazole, orphenadrine, arylacetylene, clorgyline, and diphenhydramine.

In particular embodiments, a subject combination therapy comprises administering to an individual having a disorder associated with proteinuria effective amounts of an RAR agonist and a CYP inhibitor, where the method involves administering ATRA in an amount of from about 2 mg/kg per day to about 25 mg/kg per day for the desired treatment duration; and administering the CYP inhibitor for the desired treatment duration, where the CYP inhibitor and dosage is selected from: cimetidine administered via an oral or intravenous route at a concentration in the range of about 10 mg/kg body weight to about 50 mg per kilogram of body weight, every six hours; piperine administered by an oral or intravenous route in an amount ranging from about 100 mg to about 300 mg, once daily; DEDC administered orally or intravenously at a concentration ranging from about 0.25 g/m² to about 2.5 g/m² once a day; and chlormethiazole administered orally in an amount ranging from about 0.5 g to about 3.5 g, once daily, for the desired treatment duration.

In particular embodiments, a subject combination therapy comprises administering to an individual having a disorder associated with proteinuria effective amounts of an RAR agonist and a calcium channel blocker, where the method involves administering the RAR agonist in an amount of from about 2 mg/kg per day to about 25 mg/kg per day for the desired treatment duration; and administering the calcium channel blocker in an amount of from about 1 mg to about 100 mg, where the calcium channel blocker is selected from amlodipine, bepridil, diltiazem, felodipine, flunarizine, isradipine, nicardipine, nifedipine, nimodipine, and verapamil, for the desired treatment duration.

In a particular embodiment, a subject combination therapy comprises administering to an individual having a disorder associated with proteinuria effective amounts of an RAR agonist and a calcium channel blocker, where the method involves administering ATRA in an amount of from about 2 mg/kg per day to about 25 mg/kg per day for the desired treatment duration; and administering the calcium channel blocker for the desired treatment duration, where the calcium channel blocker and dosage is selected from benazapril administered at a dosage of 10 mg orally once a day at first, which is in some cases increased to 20 mg to 40 mg orally per day taken as a single dose or divided into two doses; captopril administered at a dosage of 25 mg to 100 mg orally two or three times a day; enalapril administered at a dosage of 5 mg orally once a day at first, which is in some cases increased to 10 mg to 40 mg orally a day taken as a single dose or divided into two doses; enalapril is in some embodiments administered at a dosage of 1.25 mg intravenously every six hours; fosinopril administered at a dosage of 10 mg to 40 mg orally once a day; lisinopril is generally administered at a dosage of 10 mg to 40 mg orally once a day; and moexipril is generally administered at a dosage of 7.5 mg orally once a day at first, which is in some cases increased to 30 mg orally a day taken as a single dose or divided into two doses, for the desired treatment duration.

Subjects Suitable for Treatment

A subject treatment method is suitable for treating an individual who has been diagnosed as having proteinuria. A subject treatment method is suitable for treating an individual who has been diagnosed as having one of the following disorders: minimal change disease, focal segmental glomerular sclerosis (FSGS), membranous nephropathy, diabetic nephropathy, amyloidosis, IgA nephropathy, anti-glomerular basement membrane (anti-GBM) antibody disease, pre-eclampsia, nephrotic syndrome, fibrillary glomerulonephritis, HIV (human immunodeficiency virus) associated renal disease, HIV associated nephropathy (HIVAN), hereditary nephritis, Alport syndrome, infection related renal disease, postinfectious glomerulonephritis, membranoproliferative glomerulonephritis, lupus nephritis, Wegener's granulomatosis, microscopic polyangitis, Henoch-Schonlein purpura, vasculitis, cryoglobulinemia, post-transplant glomerulopathy, and congenital nephrosis. In some embodiments, the individual in an adult. In other embodiments, the individual is a child aged 1 year to about 5 years.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### ATRA Induces Podocyte Differentiation and Alters Nephrin and Podocin Expression In Vitro and In Vivo

Methods

Cell Culture of Rat and Mouse Podocytes

Rat Podocytes. Primary rat podocytes were phenotypically characterized using multiple podocyte specific markers, including WT-1 and synaptopodin. Rat podocytes were established in culture as previously described. Shankland et al. (1999) Kidney Int 56:538-548. In short, differential sieving was performed on isolated kidney cortex from male Sprague Dawley rats (Simonsen, Gikoy, Calif., USA) under sterile conditions. Glomeruli were collected on a sieve with 75 μm pores and placed onto culture dishes containing Vitrogen (Cohesion Technologies, Palo Alto, Calif., USA). Glomeruli were cultured in K-1 media consisting of Ham's F-12 and DME-low glucose 1:1 (Gibco, Grand Island, N.Y., USA) with 2% NuSerum (Collaborative Biomedical Products, Bedford Mass., USA) and Insulin, Transferrin and Selenium premix (Becton Dickson Labware, Bedford Mass., USA). After five to seven days in culture, colonies of podocytes growing out of glomeruli were excised and re-plated. Initially, cells were passaged every 5 days onto Vitrogen coated plates in K-1 media mixed 1:1 with 3T3 cell conditioned media. After 20 passages, cells were plated onto plastic in K-1 media alone. Podocytes were characterized by their polygonal shape and cobblestone appearance at confluency. The cells also stained positively with antibodies to FxIA, cytokeratin, and podocalyxin and stained negatively with antibodies against the mesangial cell antigen (Thy-1) and the endothelial cell antigen (Factor VIII). Johnson et al. (1992) J Am Soc Nephrol 2:1388-1397.

Mouse Podocytes.

Experiments were also performed on podocytes derived from C57/bl mice. Glomeruli were sieved and prepared as previously described. Shankland et al. (1999) Kidney Int 56:538-548; Petermann et al. (2002) Kidney Int. 61:40-50. Podocytes were grown on collagen 1 coated plates with MGEC-media containing 5% fetal bovine serum (FBS: Summit biotechnology, Ft. Collins, Colo., USA), penicillin (100 U/ml), streptomycin (100 μg/ml), and glutamine (2 mmol/L; all Irvine Scientific, Santa Ana, Calif., USA) at 37° C. in 95% air/5% CO₂. Podocytes were identified by previously described criteria (Shankland et al. (1999) supra) and by confirming the presence of podocyte specific proteins—including WT-1, nephrin, podocin, and ezrin—by immunostaining.

Immortalized Mouse Podocytes. Conditionally immortalized mouse podocytes, also termed heat sensitive mouse podocytes (HSMP), were also used in these studies. The methods for producing H-2 Kb-tsA58 transgenic mice and for isolating podocytes from the kidneys of these mice have been previously described. Jat et al. (1991) Proc Natl Acad Sci 88:5096-5100; and Mundel et al. (1997) Exp Cell Res 236:248-258. These mice are transgenic for a temperature sensitive (tsA58) SV40 large T antigen that is under control of an y-interferon (INF) inducible promoter. Cells proliferate at 33° C. with INF. Removal of INF and switching the growth temperature to 37° C. causes podocytes to stop proliferating and undertake a differentiated phenotype more closely resembling that of podocytes in vivo. For these studies, HSMP were utilized under proliferative and undifferentiated conditions in order to better demonstrate the anti-proliferative and differentiating effects of ATRA.

Exposure ofpodocvtes to ATRA. Rat podocytes, mouse podocytes, and growth permissive HSMP were plated at a concentration of 15,000 cell/cm² in standard media and allowed to adhere overnight. Culture medium was then removed, and replaced with medium containing ATRA (Sigma Chemical Co. St. Louis, Mo., USA) at concentrations of 1 μM, 5 μM, 10 μM, 50 μM, and 200 μM for 72 hours. ATRA was suspended in 100% ethanol to make a stock ATRA solution having a concentration of 10 mM ATRA, which stock solution was then diluted into media to the desired concentration. For control experiments, ethanol was diluted into standard media at similar concentrations. All experiments were handled with minimal exposure of ATRA to ambient light or air.

Cell Morphology

Cell morphology was carefully examined under light microscopy on an inverted scope. Cellular morphology was also examined by scanning electron microscopy on adherent cells fixed in half-strength Karnovsky's solution.

Immunostaining and Western Blot Analysis

Immunofluorescent staining of cultured cells. Following ATRA exposure (3 days), rat podocytes and HSMP were washed with cold phosphate buffered saline (PBS), then were fixed 100% methanol at −20° C. Shankland et al. (1997) Kidney Int. 51:1088-1099. After fixation, cells were washed again with cold PBS and were incubated with the following primary antibodies at 4° C. overnight: rabbit polyclonal to WT-1 (C-19 Santa Cruz Biotechnology, Santa Cruz, Calif., USA), rabbit polyclonal to podocin and rabbit polyclonal to synaptopodin, rabbit polyclonal to nephrin, rabbit polyclonal to p35 (C-19) (Santa Cruz), rabbit polyclonal to CD2AP and rabbit polyclonal to GLEPP1. After washing in PBS the primary antibodies were all detected using a biotinylated goat anti-rabbit antibody (Vector, Burlingame, Calif., USA) followed by an avidin conjugated AlexaFluor 594 (Molecular Probes, Eugene, Oreg., USA).

Immunofluorescent staining of kidney tissue. Mouse kidney biopsies were fixed in methyl Carnoy's (MC) solution or snap frozen in liquid nitrogen for analysis of proliferating cell nuclear antigen (PCNA), WT-1, podocin, synaptopodin, podocalyxin, and nephrin in vivo following ATRA treatment in anti-glomerular disease. Sections (4 μm) were cut from paraffin embedded MC fixed tissue or from OCT embedded frozen tissue. Frozen sections were then fixed in 100% methanol at −20° C. Mouse monoclonal antibody to PCNA (Beckman Coulter Miami, Fla., USA) or rabbit polyclonal antibody to WT-1 were incubated on MC-fixed tissue, and rabbit polyclonal antibodies to podocin, nephrin, podocalyxin, and synaptopodin were incubated on frozen sections. Primary mouse antibody to PCNA was incubated with a peroxidase conjugated rat anti-mouse IgM antibody (Zymed Laboratories, Inc. So. San Francisco, Calif., USA); all other primary antibodies were incubated with biotinylayted goat anti-rabbit antibody followed by either avidin D conjugated horseradish peroxidase (WT-1) or avidin conjugated AlexaFluor. PCNA and WT-1 stains were visualized with diaminobenzidine (Sigma Chemicals, St. Louis, Mo., USA).

Western Blot Analysis was performed as previously described. Shankland et al. (1997) Kidney Int. 51:1088-1099; Shankland et al. (1997) Kidney Int. 52:404-413; Shankland et al. (1996) Kidney Int. 50:1230-1239. Briefly, cellular protein was extracted from cultured podocytes using TG buffer containing 1% triton, 10% glycerol, 20 mmM HEPES, 100 mM NaCl (Sigma) with complete protease/phosphatase inhibitors (Roche Pharmaceuticals, Nutley, N.J., USA). Protein extracts (10 μg) were separated by SDS-PAGE gel electrophoresis and were transferred to PVDF membranes (PerkinElmer Life Sciences, Inc., Boston, Mass., USA). Membranes were blocked with 5% non-fat dried milk in Tris buffered saline TWEEN 20 non-ionic detergent and immunostained with antibodies to nephrin and podocin. To ensure equal protein loading, all western blots were also exposed to antibodies to tubulin. Specific bands were visualized using 5-bromo-4-chloro-3-indolyl phosphate/nitro blue tetrazolium (Sigma).

Measuring Cell Proliferation: MTT Assay

Podocyte activity was assessed using a 3-(4,5-dimethylthiazol-2-yl)-2,5-diphenyltetrazolium bromide (MTT) assay (Promega, Madison, Wis., USA) according to manufacturer's instructions. This assay is a non-radioactive cell proliferation assay that identifies living cells, and is based on the cellular conversion of a tetrazolium salt into a formazan product, a chromophore, which can be quantified by spectrophotometry. Briefly, cells were plated into a 96-well plate, allowed to adhere overnight; then an appropriate concentration of ATRA was added to the culture medium. Podocytes were exposed to ATRA for 3-5 days (rat podocytes or heat-sensitive mouse podocytes) or seven days (mouse podocytes). A labeling dye included in the kit was added four hours prior to the end point. A stop solution was added to each well to stop the reaction and solubilize the cells, and the absorbance was read at 570 nm.

Measuring Apoptosis: Hoechst 33342 Staining

To ensure that the anti-proliferative effects of ATRA were not confounded by podocyte apoptosis in podocytes or bi-nucleated cells, Hoechst 33342 (Molecular Probes, Eugene, Oreg., USA) staining was performed as previously reported. Hiromura et al. (1999) J Clin Invest 103:597-604. Careful morphological analysis was performed and the percentage of apoptotic cells was quantitated in adherent and non-adherent cells using inverted fluorescent microscopy as previously described. Hiromura et al. (1999) supra; and Mooney et al. (1997) J Immunol 159:3949-3960.

In Vivo Studies: Anti-Glomerular Model.

An antibody-mediated model of podocyte injury was induced in C57BL6 mice with a sheep anti-glomerular antibody (two doses of 0.3 ml/20 gm body weight) over two consecutive days. Kim et al. (1999) Kidney Int 55:2349-2361; and Ophascharoensuk et al. (1998) Kidney Int 54:416-425. After the induction of the anti-glomerular model, but before the development of proteinuria, mice were then divided into an experimental group that received daily injections of ATRA 16.6 mg/kg (IP), and a control group that received vehicle (corn oil). Experimental and control animals were studied on day 7 and 14 (n=5 per group/time point). The podocyte specific proteins WT-1, nephrin, podocin, synaptopodin, were measured by immunostaining at each time point, as was the proliferation marker, PCNA (see above). Using proliferating cell nuclear antigen (PCNA) as a marker of proliferation and WT-1 as a marker for podocytes, the level of podocyte proliferation was determined.

Renal Function Assessment. Control and experimental mice were placed in metabolic urine collection cages in order to obtain a timed collection for urinalysis. Urine was analyzed for protein content utilizing the sulphosalicylic acid (SSA) method as previously described. Shankland et al. (1996) Kidney Int. 50:116-124. Serum was also collected from mice in order to evaluate blood urea nitrogen (BUN) levels. The BUN kit (Sigma Chemicals, St. Louis, Mo., USA) is based on the direct interaction of urea with diacetyl monomoxime to produce ammonia, which is subsequently measured colorimetrically. The absorbance obtained from serum samples is compared with a standard curve of increasing concentrations of urea nitrogen standards.

Results

ATRA Reduces Podocyte Proliferation In Vitro

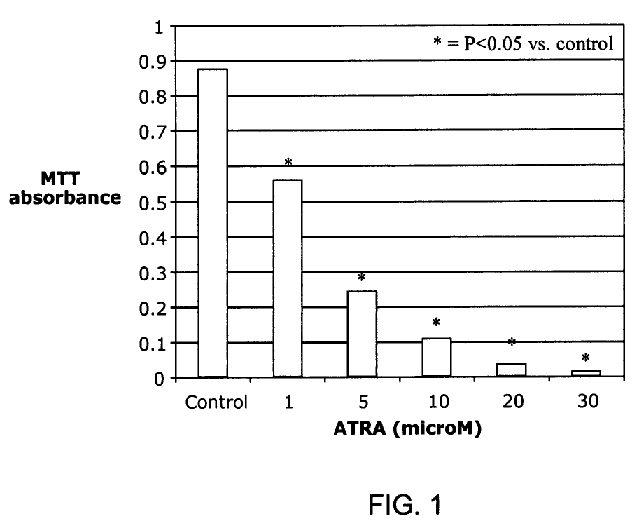

When grown in culture, all three cell types are undifferentiated and proliferate. Viable cell number was measured by MTT assay. FIG. 1 shows that ATRA reduced rat podocyte proliferation in a concentration-dependent fashion. Beginning at one μM, ATRA significantly reduced MTT absorbance compared to control cells (P<0.001). Similarly, ATRA significantly reduced MTT absorbance of mouse podocytes and heat sensitive mouse podocytes grown under growth permissive conditions although at higher concentrations of ATRA than were required with the rat podocytes.

To ensure that the decrease in cell number measured by the MTT assay was not due to a loss of cells by apoptosis or due to an increase in the number of binucleated cells, Hoechst staining was performed. The results showed that at the concentrations of ATRA used in these experiments, there was a statistically insignificant number of cells undergoing apoptosis (<4% of total cells; P<0.05). Moreover, less than 2% of cells were binucleated. Taken together, these results demonstrated that the decrease in MTT absorbance was due to an anti-proliferative effect of ATRA on the podocytes.

ATRA Induces Process Formation in Cultured Podocytes.

Electron microscopy was performed to determine the effect of ATRA on podocyte morphology. Cell morphology was assessed by scanning electron microscopy in cultured rat podocytes. There was an increase in number and length of processes in cells exposed to ATRA, with cells taking on a typical arborized phenotype. Exposing cultured rat podocytes to 1 μM ATRA resulted in the presence of processes, which extended from the cell body. Ten 1 μM ATRA caused a marked increase in the number and length of processes. Similar results were obtained in cultured HSMP.

ATRA Increases the Levels of Nephrin and Podocin In Vitro.

Both rat and heat sensitive mouse podocytes were utilized to determine if ATRA altered the expression of nephrin and podocin. Note that the concentration of ATRA required to reduce proliferation in rat podocytes was lower than concentrations required by heat sensitive mouse podocytes to achieve the same endpoint. When undifferentiated and proliferating rat podocytes were exposed to 10 μM ATRA, and when heat sensitive mouse podocytes were exposed to 200 μM ATRA, nephrin immunostaining increased, compared to control cells exposed to vehicle. Similar but less pronounced immunostaining for nephrin was seen at lower concentrations of ATRA for both cells types, suggesting a titrational effect of the ATRA concentration.

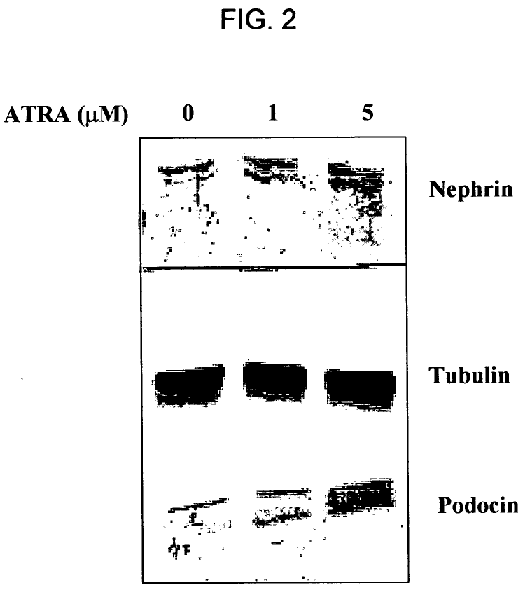

Similarly, increased immunostaining was observed for podocin in cultured rat podocytes given OptM ATRA and in HSMP given 200 μM ATRA, compared to control. Western blot analysis was used to confirm these findings in primary mouse podocytes, and the results are shown in FIG. 2. ATRA at 1 μM and 5 μM increased the protein levels for nephrin and podocin in the primary mouse cultures. Taken together, these results show that ATRA increased the protein levels for nephrin and podocin in cultured podocytes in a concentration dependent fashion, and this coincided with a decrease in proliferation.

Proteinuria is Reduced in Experimental Glomerular Disease by ATRA.

Pilot studies were performed to determine if giving ATRA 16 mg/kg i.p. for 7 days caused adverse side effects in normal mice. The results showed that there was no weight loss, ruffled fur, withdrawal or diarrhea when administered to mice at this concentration. To determine the effect of ATRA on podocytes in vivo, mice with experimental glomerular disease were given ATRA intraperitoneally (i.p.) on two consecutive days. ATRA did not interfere with the binding of the anti-glomerular antibody used to induce disease. Renal function was assessed by measuring proteinuria and BUN.

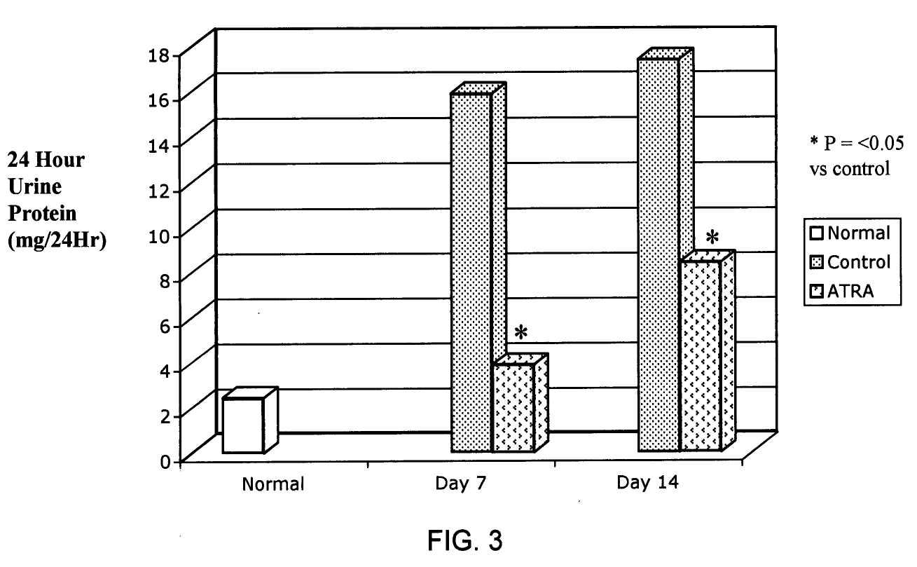

FIG. 3 shows that, compared to normal mice, proteinuria increased significantly in control mice given the anti-glomerular antibody and vehicle on day 7 (P<0.001 vs normal) and day 14 (P<0.001 vs normal). ATRA significantly reduced proteinuria in mice with anti-glomerular disease at day 7 (P<0.001 vs control) and day 14 (P<0.001 vs control). BUN increased in control rats at days 7 (P<0.01 vs normal) and 14 (P<0.01 vs normal), but this was not altered by the administration of ATRA.

FIG. 3. ATRA reduces proteinuria in experimental glomerular disease. Podocyte injury was induced in mice with an anti-glomerular antibody, and proteinuria was measured in control (vehicle treated) and experimental (ATRA treated) animals at days 7 and 14. There was a significant increase in proteinuria in control mice with disease. ATRA significantly reduced proteinuria at days 7 and 14 in experimental mice with disease.

ATRA Prevents a Decrease in Nephrin and Podocin in Mice with Glomerular Disease.

Immunostaining for the slit diaphragm proteins, nephrin and podocin, was analyzed in normal, control and experimental mice on days 5 and 14 after disease induction. Experimental glomerular disease caused a decrease in immunostaining for synaptopodin, podocin, nephrin, p35, and CD2AP in control nephritic mice given vehicle. In contrast, ATRA prevented the decrease in immunostaining for synaptopodin, podocin, and nephrin in the experimental group of nephritic mice. The effects of ATRA on p35v and Cd2AP were not different from control nephritic animals given vehicle. Taken together, in this model, ATRA treatment prevented the decrease in immunostaining of the slit diaphragm proteins nephrin and podocin, and also the actin-binding proteins, synaptopodin, with a concomitant reduction in proteinuria.

Podocyte Proliferation and Differentiation are Altered by ATRA in Experimental Glomerular disease.

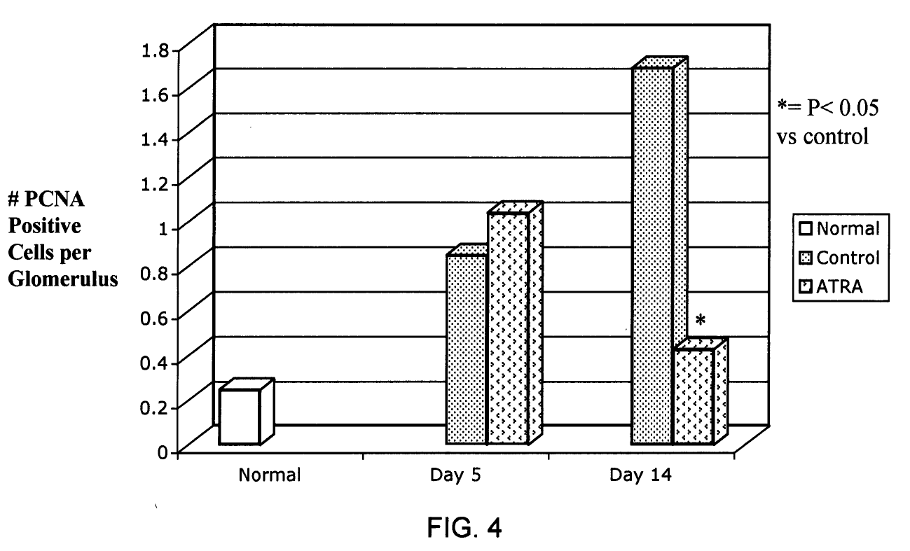

It has previously been shown that giving the anti-glomerular antibody to mice causes podocyte de-differentiation and increased proliferation. Kim et al. (1999) supra. Immunostaining for PCNA was used as a marker for DNA synthesis. FIG. 4 shows the number of PCNA positive cells in the ATRA treated and control groups at days 7 and 14. PCNA staining was significantly increased at day 7 (P<0.05 vs. normal) and day 14 (P<0.05 vs. normal) in control mice given the anti-glomerular antibody and vehicle. ATRA did not have a statistically significant effect on PCNA staining on day 7. In contrast, ATRA significantly reduced podocyte proliferation in nephritic mice at day 14 (P<0.01 vs. control).

FIG. 4. A TRA alters podocyte proliferation in experimental glomerular disease. Podocyte proliferation was measured by immunostaining for proliferating cell nuclear antigen (PCNA) in control and experimental mice. The number of cells that stained positive for PCNA increased in control and experimental mice at day 5 (P<0.05 vs normal). PCNA staining increased further at day 14 in control mice (P<0.001 vs day 5). ATRA significantly reduced PCNA immunostaining in experimental mice at day 14 (P<0.001 vs control).

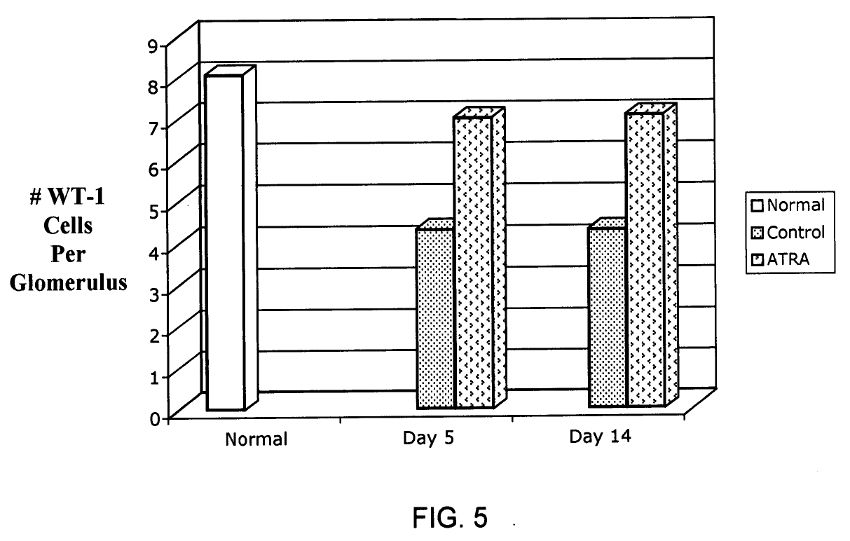

It has been shown that podocytes de-differentiate in order to proliferate. Kim et al. (1999) supra; Shankland et al. (2000) Kidney Int 58:674-683; Barisoni et al. (1999) J Am Soc Nephrol 10:51-61. It has been shown that WT-1 staining is specifically increased in differentiated podocytes. Kreidberg et al. (1993) Cell 74:679-691. To determine if the decrease in podocyte proliferation by ATRA was due to the prevention of podocyte de-differentiation, WT-1 staining was performed in control and experimental mice, and the results are shown in FIG. 5. Quantitation showed a significant decrease in WT-1 staining in control mice compared to normal at day 7 (P<0.001) and day 14 (P<0.001). In contrast, giving ATRA to nephritic mice prevented the decrease in WT-1 staining at day 7 (P<0.001 vs control) and day 14 (P<0.001 vs control) of nephritis.

FIG. 5. WT-1 immunostaining. Podocyte number was evaluated quantitating the number of cells expressing WT-1. Podocyte number decreased in control animals with anti-glomerular antibody disease given vehicle at days 7 and 14. ATRA prevented the decrease in podocyte number at both time points.

### Example 2

### ATRA Improves HIV-Associated Nephropathy

Materials and Methods

In Vitro Studies

Study Groups

To test the hypothesis that ATRA altered the proliferative capacity of podocytes in HIV transgenic mice, conditionally immortalized podocytes harvested from transgenic HIV Tg-26 (Tg) and conditionally immortalized podocytes from transgene free or wild-type podocytes (controls) were compared in a series of studies. Both transgenic and wild-type cell lines were initially plated in “growth permissive” conditions (33° C.) and grown in 10 ml of “standard media,” consisting of 90% RPMI 1640 (Invitrogen, Grand Island, N.Y.), 10% fetal bovine serum (Hyclone, Logan, Utah), 1% L-glutamine (Irvine Scientific, Santa Ana, Calif.), 1% pen-strep (Biosource, Rockville, Md.), 1% sodium bicarbonate (Sigma-Aldrich, St Louis, Mo.) and 1% HEPES buffer (Sigma-Aldrich, Santa, Ana, Calif.), and 1% sodium pyruvate (Irvine Scientific, Santa Ana, Calif.). For growth promotion, 5 μl of mouse gamma interferon were added to 10 ml of media. After cells reached 95% confluence, they were passaged and plated into in 24-well plates and incubated at 37° C. in the absence of interferon in the media; incubation at 37° C. in medium without interferon is referred to as “growth restrictive” conditions.

At the initiation of growth restriction, transgenic and wild type cells were each separated into an experimental and control group. Experimental podocytes grown in 24-well plates containing 1 ml standard media plus 25 mMol/ml ATRA (Sigma Laboratories) dissolved in a 3 mg/ml solution of 100% ethyl alcohol. The control group was exposed to standard media. Taken together, there were four study groups: (1) wild-type podocytes in standard media (WT), (2) transgenic podocytes in standard media (Tg), (3) wild-type podocytes in standard media plus 25 mMol/ml ATRA WT ATRA) and (4) transgenic podocytes exposed to standard media plus 25 mMol/ml ATRA (Tg ATRA).

The dose of 25 mMol/ml ATRA was selected after exposing cells to varying mMol doses of ATRA (10, 25, 50, 75, 100) and then measuring apoptosis with the Hoecht's Assay according to the manufacturer's directions. Apoptosis was not increased at 25 mM.

Each study group was assessed for proliferation (see below) at three different time points as follows: (i) day “1” of growth restriction which was defined as 24 h after plating cells into growth restrictive conditions; (ii) day 7 of growth restrictive conditions. Cells were exposed to ATRA or control conditions for days 3-7. (iii) day 14 of growth restrictive conditions, where ATRA or control was added to the media for days 10-14.

Measuring Proliferation

Cell proliferation was assessed by the MTT (Promega, Madison, Wis.) assay on days 1, 7, and 14 of growth restriction. In an effort to minimize dye usage, the volume/well was reduced from 1 ml to 300 μl/well in all groups. A volume of 45 μl/well of MTT dye solution was added to each well. The remainder of the assay was performed according to the manufacturer's specifications.

Cellular proliferation was also assessed through direct visualization of 10 cm plated cells at d 1, 2, 4, 6 and 8 growth restriction. Proliferation was assessed by the number of cells seen within a small circle marked on the plate. A total of four circles were marked and observed per plate. The circles were numbered #1-4 and photographed at the above time points. One exemplary circle was chosen per plate and followed through all time points. The four groups were compared at all time points.

Immunostaining

To determine the effect of ATRA on podocyte differentiation and mechanisms governing proliferation, the protein level of podocyte specific proteins and cell cycle proteins was assessed by immunostaining. Cells were washed with cold phosphate buffered saline (PBS)×3 and fixed with methanol. A primary antibody was then placed on the fixed cells All primary antibodies (Ab) were dissolved in a 250 μl/well solution of 1% bovine serum albumin (BSA) in PBS at the following concentrations: NO primary Ab (control); anti-Nephrin Ab (Research Diagnostics, Inc, Flanders, N.J.) at 1/500 dilution; anti-WT-1 Ab (Santa Cruz Biotechnology, Inc, Santa Cruz, Calif.), 1/250 dilution; Podocin, 1/250 dilution; anti-Ezrin Ab, 1/500 dilution; and anti-cd2ap Ab. Cell cycle proteins were also assessed. The same solution was used and antibody concentrations were as follows: anti-p21 Ab (BD PharMingen, SanDiego, Calif.) 1/100, anti-p27 Ab (BD Transduction Laboratories, San Diego, Calif.) 1/100 and anti-cyclin A Ab (Santa Cruz Biotechnology, Inc, Santa Cruz, Calif.) 1/300.

The primary antibody was incubated with the fixed cells overnight at 4° C. A Biotinylated secondary antibody was then applied to all wells using the same solution. Most primary antibodies called for a goat anti-rabbit IgG (Vector Laboratories, Burlingame, Calif.) secondary antibody. However, the anti-p21 and anti-p27 Abs required a horse anti-mouse antibody (Vector Laboratories, Burlingame, Calif.) at 1/500 and 1/200 dilutions, respectively. The secondary antibody was left on the fixed cells for 30 minutes at room temperature. A Strepavidin Alexa Fluor (Molecular Probes, Inc, Eugene, Oreg.) fluorescent tertiary antibody was applied to all wells at 1/200 dilution in the same solution for 30 minutes at room temperature. DAPI fixative (Vector Laboratories, Burlingame, Calif.) was applied to all wells. After fixation, photographs were obtained for comparison.

Western Blot Analysis

Western blot analysis was also used to measure differences in podocyte proteins and cell cycle proteins. Cells from day 1, 4 days, and 8 days of growth restriction were washed with cold PBS, trypsinized, and suspended in standard media. After centrifugation, cells were transferred to a microcentrifuge, and resuspended in cold PBS for a second centrifugation. Supernatant was discarded and TG buffer (100 cc=HEPES 0.52 g, NaCl 0.584 g, Triton 100× 1 cc, Glycerol 10 cc, H₂O 98 cc) in 2× volume of cell pellet was added to microfuge tube. Cells were frozen overnight at −70° C. to disrupt cell membranes.

Protein concentration was measured by bicinchoninic acid (BCA), according to the manufacturer's instructions (Pierce, Rockford, Ill.). Set volumes of protein in buffer solution and reagents were measured spectrographically. These values were then compared to a standard scale to establish protein content by weight (pg).

Five μg of protein was loaded onto a 12% Gel. After transfer of the proteins to a membrane, the membrane was exposed to diluted antibody for p21 (1/100 dilution), p27 (1/100) and cyclin A (1/300). The membrane was then developed and bands were compared. Tubulin was used as a housekeeper protein for the p21 and p27 bands, and GAPDH was used for cyclin A.

In vivo Studies

Experimental Design

A total of six breeder Tg-26 HIV transgenic heterozygous mice were used. These mice were originally developed through incorporation of a truncated form of the HIV genome into fertilized eggs of the FVB/N mouse. Dickie et al (1991) Virology 185:109-119. Due to variable penetrance of the transgene, the offspring were genotyped by polymerase chain reaction (PCR) to determine the presence or the absence of HIV transgene (Tg). Littermates were divided into two groups: (i) controls: five wild-type (WT) and 5 transgenic (Tg) were given intraperitoneal injections of corn oil and (ii) Experimental group: 2 WT and 5 Tg, were given a solution of corn oil and ATRA. The ATRA powder was dissolved in corn oil in 3 mg/ml solution. Both the corn oil and ATRA/corn oil solution was given in doses of 16 mg/kg three times a week. Injections were initiated at ten days and carried through 4 months of life at which time animals were sacrificed.

Urine and serum were collected at 20-day intervals. Urine was assayed for protein using the SSA method and creatinine using a colorimetric assay. Serum was assayed for BUN using a spectrographic assay (Sigma).

Kidney tissue from sacrificed animals (see time point at end of 1^{st} experimental design paragraph) was stained for PAS (periodic acid Schiff) and hematoxylin for light microscopic analysis. To assess the level of disease, a glomerulosclerosis index and interstitial disease index were measured on tissue of all animals. A total of 50 glomeruli were observed per tissue sample and the percentage of segmental sclerosis was recorded as 0, 0-25%, 25-50%, 50-75% or 75-100%. Collapsed glomerular tufts were also recorded. The degree of interstitial disease (primarily tubular dilatation) was recorded as an overall impression of the biopsy with the same percentage range.

Immunohistochemical analysis for staining for WT-1 was used to further confirm podocyte proliferation in the disease model. The number of podocytes/glomerulus was approximated via number of WT-1 positive nuclei in a podocyte distribution in total of 10 glomeruli per animal.

Statistical Analysis

The student t test was used for statistical analysis. A p value of less than 0.05 was considered statistically significant.

Results

In Vitro Studies

Growth Studies

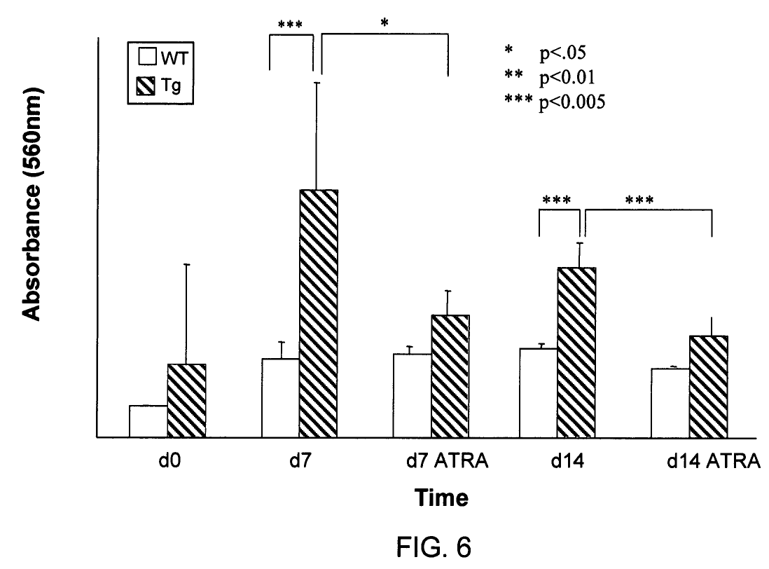

Cellular proliferation was measured by MTT assay. As previously described, the proliferative capacity of HIV transgenic podocytes exposed to standard media (Tg) was three times that compared to wild-type (WT) podocytes at day 7(p<. 005) (FIG. 6). At 14 days, there was two fold increase in proliferation in the transgenic (Tg) podocytes compared to WT (p<. 005). FIG. 6 shows that when ATRA was added to the media, the proliferation of transgenic podocytes (Tg ATRA) was reduced by 50% and 25% at 7 (p<. 05) and 14 days (p<0.005), respectively, compared to Tg podocytes incubated with medium without ATRA. When WT podocytes without ATRA are compared to WT podocytes exposed to ATRA there is no measurable difference. These data suggest that ATRA is not toxic to the podocyte; and that ATRA reduces proliferation of podocytes from the Tg mouse.

FIG. 6: In the absence of ATRA, proliferation (assessed by MTT assay) was increased 3× and 2× as compared to WT at 7d and 14d growth restriction respectively. ATRA reduced proliferation by 50% at d7 and by 25% at dl4.

Sequential photographs of transgenic and wild type podocytes in 10 cm cultures dishes also demonstrate greater proliferation in the transgenic podocytes, which proliferation is attenuated by the addition of ATRA.

Immunoflorescence/Westemblot:

Transgenic podocytes at day 7 of growth restriction, when exposed to ATRA, exhibited increased intensity of staining for the podocyte slit diaphragm protein cd2ap when compared to transgenic podocytes at day 7 of growth restriction in standard media. The cells also appeared larger and decreased in number when exposed to ATRA. Immunostaining at day 14 of growth restriction showed increased staining intensity for CD2AP in the transgenic ATRA versus standard media group. The decrease in cell number was not as prominent at the day 14 day time point.

The podocyte transcription regulator protein WT-1 also showed increased staining intensity, increased cell size and decreased cell number at day 7 of growth restriction in the transgenic ATRA group versus standard media.

The cell cycle dependent protein cyclin A, known to promote progression of the cell cycle, showed decreased intensity of staining in the ATRA exposed group as compared to standard media. Cell number was also grossly decreased in the ATRA group.

In Vivo Studies

Proteinuria

The carrier-treated transgenic versus wild-type mice tended to have more proteinuria at all time points. This difference, however, was not statistically significant because of the great variability (large standard deviation) of urinary protein excretion from one animal to the next.

The transgenic mice that were given intraperitoneal injections of ATRA tended to have less proteinuria versus carrier treated animals (Tg C). This difference was statistically significant at 80 days. All other time points were not significant, again because of great variability.

Serum Studies:

There was no significant difference in serum BUN in the different groups.

Histopathology:

Results of glomerulosclerosis grading are averages of the different groups. Comparisons were made between the carrier treated WT (2 animals) and Tg (5 animals) and the transgenic carrier versus ATRA treated (5 animals). In general there was greater segmental sclerosis in the carrier treated transgenic animals versus carrier treated wild type. The addition of ATRA reduced the sclerosis by a statistically significant amount. The details are as follows; 86% of carrier treated WT glomeruli (50 Total) where without sclerosis, 13% had 0-25% and 1% had 25-50% sclerosis. There were no glomeruli with 50-75% or 75-100% sclerosis. There were no collapsed glomeruli. 74.2% of the carrier treated Tg glomeruli has no sclerosis, 16.8 had 0-25%, 7.2% had 25-50%, 0.4% 50-75%, 0% 75-100% and 0.4% collapsed. No statistical significance was seen in the comparison of % segmental sclerosis groups. When ATRA was added the Tg mice had 94% with no sclerosis, 4.8% with 0-25% and 1.2% with 25-50 sclerosis. No glomeruli were seen with 50-75% or 75-100% sclerosis. There were no collapsed glomeruli. Again, difference between the carrier and ATRA treated group was significant.

Interstitial disease is reported as an overall impression of the biopsy sample. Generally the level of interstitial disease was minimal is most animals of all groups. The usual score was 0-25% tubular dilatation. An exception was a biopsy of a carrier treated transgenic mice where there was significant segmental sclerosis. The interstitial disease score in this case was a clear 25% tubular dilatation. The other exceptions were two ATRA treated Tg animals that had no tubular dilatation.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of reducing proteinuria in an individual, the method comprising administering to the individual an effective amount of a retinoic acid receptor (RAR) agonist or a retinoid X receptor (RXR) agonist.

2. The method of claim 1, wherein the RAR agonist is all trans retinoic acid (ATRA).

3. The method of claim 2, wherein the ATRA is administered in an amount of from about 2 mg/kg body weight to about 50 mg/kg body weight.

4. The method of claim 1, wherein the RAR agonist or the RXR agonist is administered orally.

5. The method of claim 1, wherein the RAR agonist or the RXR agonist is administered intravenously.

6. The method of claim 1, further comprising administering a second therapeutic agent selected from an angiotensin converting enzyme inhibitor, an angiotensin receptor blocker, a cortisosteroid, a calcium channel blocker, a cytochrome P450 inhibitor, cyclophosphamide, cyclosporine, tacrolimus, mycophenolate, azathioprine, a non-steroidal anti-inflammatory drug, rituximab, intravenous immunoglobulin, an anti-C5 monoclonal antibody, levamisole, and an omega-3 fatty acid.

7. The method of claim 1, wherein the proteinuria results from a disorder selected from nephrotic syndrome, minimal change disease, focal segmental glomerular sclerosis, membranous nephropathy, diabetic nephropathy, amyloidosis, IgA nephropathy, anti-glomerular basement membrane antibody disease, pre-eclampsia, human immunodeficiency virus-induced nephropathy, nephrotic syndrome, infectious related kidney disease, postinfectious glomerulonephritis, lupus nephritis, and congenital nephrosis.

8. A method of treating nephrotic syndrome in an individual, the method comprising administering to the individual an effective amount of a retinoic acid receptor (RAR) agonist or a retinoid X receptor (RXR) agonist.

9. The method of claim 8, wherein the RAR agonist is all trans retinoic acid (ATRA).

10. The method of claim 9, wherein the ATRA is administered in an amount of from about 2 mg/kg body weight to about 50 mg/kg body weight.

11. The method of claim 8, wherein the RAR agonist or the RXR agonist is administered orally.

12. The method of claim 8, wherein the RAR agonist or the RXR agonist is administered intravenously.

13. The method of claim 8, further comprising administering a second therapeutic agent selected from angiotensin converting enzyme inhibitor, an angiotensin receptor blocker, a cortisosteroid, a calcium channel blocker, a cytochrome P450 inhibitor, cyclophosphamide, cyclosporine, tacrolimus, mycophenolate, azathioprine, a non-steroidal anti-inflammatory drug, rituximab, intravenous immunoglobulin, an anti-C5 monoclonal antibody, levamisole, and an omega-3 fatty acid.
